(19) United States
(12) Patent Application Publication
(10) Pub. No.: US 2004/0068127 A1
(43) Pub. Date: Apr. 8, 2004

(54) Method for making an oxirane

(76) Inventors: Jean-Paul Schoebrechts, Grez-Doiceau (BE); Michel Strebelle, Brussels (BE); Jean-Pierre Catinat, Waudrez (BE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/220,277

(22) PCT Filed: Feb. 23, 2001
(86) PCT No.: PCT/EP01/02139

(30) Foreign Application Priority Data
Mar. 10, 2000 (FR) 00/03207

(51) Int. Cl. C07D 301/12

(52) U.S. Cl. 549/531

(57) ABSTRACT

Process for manufacturing an oxirane, in which an olefin is reacted, in a diluent chosen from water, alcohols and ketones, with a peroxide compound in the presence of a catalyst based on titanium silicalite of TS-1 type and in the presence of a nitrile.

The invention relates to a process for manufacturing an oxirane by reaction between an olefin and a peroxide compound in the presence of a catalyst and a diluent. The invention relates more particularly to a process for manufacturing 1,2-epoxypropane (propylene oxide) or 1,2-epoxy-3-chloropropane (epichlorohydrin) by reaction between propylene or allyl chloride and hydrogen peroxide.

It is known practice to manufacture propylene oxide by epoxidizing propylene using hydrogen peroxide in a solvent and in the presence of a catalyst of TS-1 type, as disclosed, for example, in patent application EP-A-0 568 336. Methanol is used as solvent in the examples.

This known process has the drawback of resulting in the formation of by-products. Specifically, when propylene oxide is manufactured, by-products are formed by reaction between the propylene oxide and water or methanol, and in particular propylene glycol and methoxypropanols of formulae CH₃—CHOH—CH₂—OCH₃ and CH₃—CH(OCH₃)—CH₂OH. When epichlorohydrin is manufactured, by-products are formed by reaction between the epichlorohydrin and water or methanol, and in particular 1-chloropropanediol and chloromethoxypropanols of formulae ClCH₂—CHOH—CH₂—OCH₃ and Cl—CH₂—CH(OCH₃)—CH₂OH. The formation of by-products reduces the selectivity of the process and consequently its yield.

The invention is directed towards preventing the formation of by-products and thus towards providing a highly selective process, while at the same time maintaining high activity (or a high reaction rate).

The invention consequently relates to a process for manufacturing an oxirane, in which an olefin is reacted, in a diluent chosen from water, alcohols and ketones, with a peroxide compound in the presence of a catalyst based on titanium silicalite of type TS-1 and in the presence of a nitrile.

One of the essential characteristics of the invention lies in the presence of nitrile in the epoxidation medium. Specifically, it has been found that the presence of a nitrile, even at low content, makes it possible to greatly reduce the formation of by-products such as methoxypropanols. For example, by adding a nitrile to the epoxidation medium, the amount of by-products formed can be reduced compared with a process performed under identical conditions but in the absence of nitrile, by at least 20%, in particular by at least 30%, preferably by at least 50%. In certain cases, the amount of by-products may be reduced by at least 75%. A selectivity towards epoxide, expressed by the molar ratio of the epoxide formed to the sum of the by-products (expressed as C3) plus the epoxide, of at least 75%, in particular of at least 80% and preferably of at least 85%, may thus be expected, a selectivity of at least 90% being particularly preferred.

The amount of nitrile used in the process according to the invention may vary within a wide range. Very low doses already have a significant effect on the formation of by-products. Excessively large amounts may not be desirable in certain cases, since they result in a reduction of the reaction rate. In general, a good compromise between the rate and the selectivity is obtained with a molar ratio of the amounts of nitrile and of diluent used of at least 0.001%. This ratio is in particular at least 0.005%, preferably at least 0.01%. A ratio of at least 0.1% gives the best results. The ratio is usually not more than 50%, in particular not more than 45%, more particularly not more than 40%. This ratio can be for example less than 34%, in particular not more than 30%. A ratio of not more than 10% is preferred. A ratio of not more than 5% gives good results.

The amount of nitrile used in the process according to the invention is usually such that the molar ratio nitrile/olefin is at least 0.00003, in particular at least 0.0003 and preferably at least 0.003. This ratio is habitually not more than 1.35, in particular not more than 0.9 and preferably not more than 0.15.

The nitrile used in the process according to the invention may be chosen from linear or branched, saturated aliphatic nitrites and from aromatic nitrites. Saturated aliphatic nitrites are preferred. Generally, the nitrile contains up to 10 carbon atoms, preferably from 2 to 7 carbon atoms. Nitriles which may be mentioned include acetonitrile and pivalonitrile. Acetonitrile is preferred. Nitriles which form an azeotrope with the diluent, such as acetonitrile with methanol, have the advantage of being easy to recycle with the diluent.

The diluent used in the process according to the invention is in most cases organic. It may be chosen from linear or branched, saturated aliphatic alcohols. The alcoholic diluent generally contains up to 10 carbon atoms, preferably from 1 to 6 carbon atoms. Examples which may be mentioned include methanol and ethanol. Methanol is preferred.

The epoxidation medium in which the olefin reacts with the peroxide compound in the presence of the catalyst, the alcohol diluent and the nitrile usually also contains water. The epoxidation medium generally comprises a liquid phase, a gaseous phase and the catalyst in solid form. The liquid phase contains the diluent, the nitrile, the dissolved olefin, the peroxide compound, a fraction of the epoxide formed and water.

The total amount of diluent and nitrile used in the process according to the invention is generally at least 35% by weight of the liquid phase defined above, in particular at least 60% by weight, for example at least 75% by weight. This amount usually does not exceed 99% by weight and in particular does not exceed 95% by weight.

The molar ratio between the amounts of olefin and of peroxide compound used in the process according to the invention is generally at least 0.1, in particular at least 1 and preferably at least 5. This molar ratio is usually not more than 100, in particular not more than 50 and preferably not more than 25.

The process according to the invention may be continuous or batchwise.

In the process according to the invention, when it is performed continuously, the peroxide compound is generally used in an amount of at least 0.005 mol per hour and per gram of titanium silicalite, in particular of at least 0.01 mol per hour and per gram of titanium silicalite. The amount of peroxide compound is usually less than or equal to 2.5 mol per hour and per gram of titanium silicalite and in particular less than or equal to 1 mol per hour and per gram of titanium silicalite. Preference is shown for an amount of peroxide compound of greater than or equal to 0.03 mol per hour and per gram of titanium silicalite and less than or equal to 0.25 mol per hour and per gram of titanium silicalite.

In the process according to the invention, the peroxide compound is advantageously used in the form of an aqueous solution. In general, the aqueous solution contains at least 10% by weight of peroxide compound, in particular at least 20% by weight. It usually contains not more than 70% by weight of peroxide compound, in particular 50% by weight.

The temperature of the reaction between the olefin and the peroxide compound may range from 10° C. to 100° C. In one advantageous variant, it is greater than 35° C. to overcome the gradual deactivation of the catalyst. The temperature may be greater than or equal to 40° C. and preferably greater than or equal to 45° C. A temperature of greater than or equal to 50° C. is most particularly preferred. The reaction temperature is preferably less than 80° C.

In the process according to the invention, the reaction between the olefin and the peroxide compound may take place at atmospheric pressure. It may also take place under pressure. This pressure generally does not exceed 40 bar. A pressure of 20 bar is suitable in practice.

The peroxide compounds which may be used in the process according to the invention are peroxide compounds containing one or more peroxide functions (—OOH) which may release active oxygen and which are capable of carrying out an epoxidation. Hydrogen peroxide and peroxide compounds which may produce hydrogen peroxide under the conditions of the epoxidation reaction are suitable for use. Hydrogen peroxide is preferred.

When hydrogen peroxide is used, it may be advantageous to use, in the process according to the invention, an aqueous hydrogen peroxide solution in crude form, i.e. in unpurified form. For example, a solution obtained by simple extraction with substantially pure water of the mixture derived from the oxidation of at least one alkylanthrahydroquinone (process known as “autoxidation AO process”) may be used without a subsequent washing and/or purification treatment. These crude hydrogen peroxide solutions generally contain from 0.001 to 10 g/l of organic impurities expressed as TOC (Total Organic Carbon) They usually contain metal cations (such as alkali metals or alkaline-earth metals, for instance sodium) and anions (such as phosphates or nitrates) in contents of from 0.01 to 10 g/l.

The oxirane which may be prepared by the process according to the invention is an organic compound comprising a group corresponding to the general formula

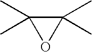

The oxirane generally contains from 3 to 10 carbon atoms, preferably from 3 to 6 carbon atoms. The oxiranes which may be prepared advantageously by the process according to the invention are 1,2-epoxypropane and 1,2-epoxy-3-chloropropane.

The olefins which are suitable in the process according to the invention generally contain from 3 to 10 carbon atoms and preferably 3 to 6 carbon atoms. They are preferably non aromatic. Propylene, butylene and allyl chloride are suitable for use. Propylene and allyl chloride are preferred.

The titanium silicalite of TS-1 type used in the process according to the invention is a titanium zeolite consisting of silicon oxide and titanium oxide and having a crystal structure of ZSM-5 type. The titanium silicalite generally has an infrared absorption band at about 950-960 cm⁻¹. Titanium silicalites corresponding to the formula xTiO₂(1−x)SiO₂ in which x is from 0.0001 to 0.5 and preferably from 0.001 to 0.05 give good results.

In the process according to the invention, a gas which has no negative effect on the epoxidation reaction may also be added to the reactor. Specifically, in patent application WO 99/48883 (the content of which is incorporated by reference into the present patent application), the Applicant found that by introducing a gaseous compound into the reaction medium at a flow rate which is sufficient to allow the oxirane produced to be entrained and removed from the reactor at the same time as the gaseous compound, the contact time between the oxirane produced and the epoxidation reaction medium is reduced. The formation of by-products is thus also prevented and the selectivity towards epoxidation is increased.

In the process according to the invention, any type of reactor may be used, in particular a reactor of loop type. Reactors of loop type with a bubble siphon, in which the circulation of the liquid and optionally also of the catalyst is obtained by bubbling a gas into one of the arms are suitable for use. This type of reactor is disclosed in patent application WO 99/48883 mentioned above.

In the process according to the invention, it may prove to be advantageous to monitor the pH of the liquid phase. For example, it may be advantageous to maintain the pH of the liquid phase during the reaction between the olefin and the peroxide compound at a value of from 4.8 to 6.5, for example by adding a base (sodium hydroxide) to the epoxidation medium, as recommended in patent application WO 99/48882 by the Applicant (the content of which is incorporated by reference into the present patent application).

The reaction between the olefin and the peroxide compound may be carried out in the presence of a salt such as sodium chloride, as disclosed in patent application WO EP 99/08703 by the Applicant (the content of which is incorporated by reference into the present patent application).

It may be advantageous to introduce the olefin into the reactor, in which the epoxidation reaction takes place, in a form diluted in one or more alkanes. For example, a fluid containing the olefin and at least 10% (in particular 20%, for example at least 30%) by volume of one or more alkanes may be introduced into the epoxidation reactor. For example, in the case of propylene, this may be mixed with at least 10% by volume of propane when the recycled unconverted propylene is introduced into the reactor. It may also be a source of propylene which is not completely freed of propane.

### EXAMPLE 1

### Not in Accordance with the Invention

0.97 g of TS-1 and 38.4 g of methanol (1.2 mol) are introduced into a jacketed Pyrex reactor equipped with a paddle stirrer and on which is mounted a condenser cooled to −20° C. The suspension is stirred at 750 rpm and the temperature is set at 25° C. Propylene is then introduced at a flow rate of 6 Nl/h via, a sintered tube. After flushing for 30 minutes with propylene (Pe), 7.4 g of a 34.4 wt % hydrogen peroxide solution (0.075 mol) are added over 20 min.

The hydrogen peroxide content is determined by iodometry after 90 min. The propylene oxide (PO) content of the gaseous phase is measured in-line by gas chromatography. The liquid phase containing the propylene oxide and the by-products (methoxypropanols “MeOPols” and propylene glycol “Diol”) is analysed by gas chromatography at the end of the test. The results are collated in Table 1.

### EXAMPLES 2 to 4

### In Accordance with the Invention

The process is performed as in Example 1, except that the solvent consists of a mixture of methanol (MeOH) and acetonitrile (MeCN). The results are collated in Table 1.

TABLE 1

| | | Degree of | Selectivity | Content of |
|---|---|---|---|---|
| | MeOH/MeCN | conversion of | towards | by-products |
| | molar | H₂O₂ after | epoxide | MeOPols + Diol |
| Ex. | ratio | 90 mm (mol %) | (mol %) | (mol %) |
| 1 | 100/0 | 99.6 | 83.7 | 16.3 |
| 2 | 99.8/0.2 | 98.9 | 87.4 | 12.6 |
| 3 | 99/1 | 98.7 | 91.3 | 8.7 |
| 4 | 95/5 | 94.3 | 94.9 | 5.1 |

The presence of acetamide in the reaction medium at the end of the reaction has not been detected.

### EXAMPLE 5

### In Accordance with the Invention

The process is performed as in Example 1, except that the solvent consists of a mixture of methanol and pivalonitrile in a ratio of 99/1 mol/mol. After reaction for 90 min, the degree of conversion of the peroxide is 92.6 mol %. The selectivities are, respectively, 92.2 mol % (propylene oxide) and 7.8 mol % (methoxypropanols and propylene glycol).

The presence of acetamide in the reaction medium at the end of the reaction has not been detected.

### EXAMPLE 6

### Not in Accordance with the Invention

1.20 g of TS-1, 38.4 g of methanol (1.2 mol) and 11.5 g of allyl chloride (0.15 mol) are introduced into the reactor of Example 1. The suspension is stirred at 750 rpm and the temperature is set at 50° C. 6.6 g of a 38.6 wt % hydrogen peroxide solution (0.075 mol) are then introduced over 20 min.

The hydrogen peroxide content is determined by iodometry after 30 min. The liquid phase containing the epichlorohydrin (EPI) and the by-products (chloromethoxypropanols and chloropropanediol) is analysed by gas chromatography at the end of the test.

After reaction for 30 min, the degree of conversion of the peroxide is 100 mol %. The selectivities are, respectively, 98.4 mol % (epichlorohydrin) and 1.6 mol % (chloromethoxypropanols and chloropropanediol).

### EXAMPLE 7

### In Accordance with the Invention

The process is performed as in Example 6, except that the solvent consists of a mixture of methanol and acetonitrile in a ratio of 99/1 mol/mol. After reaction for 30 min, the degree of conversion of the peroxide is 100 mol %. The selectivities are, respectively, 99.6 mol % (epichlorohydrin) and 0.4 mol % (chloromethoxypropanols and chloropropane diol).

The presence of acetamide in the reaction medium at the end of the reaction has not been detected.

### EXAMPLES 8 to 10

### In Accordance with the Invention

4.5 g of silica beads 0.4-0.6 mm in diameter containing 1.5 g of TS-1 are introduced into a continuous reactor of the loop type with a bubble siphon as disclosed in patent application WO 99/48883. The flow rate of H₂O₂ used in the form of a 39% aqueous solution is 0.174 mol/h. A continuous feed of diluent (=methanol) is provided to maintain a ratio CH₃OH/H₂O₂=16 mol/mol.

According to the example (see Table 2), this CH₃OH is added in pure form or supplemented with 0.3% or 1% of acetonitrile.

The temperature of the tests is maintained at 55° C. A high flow rate of Pe is used (19.6 mol/mol of H₂O₂, i.e. 75 Nl/h). This gas makes it possible, via the bubble-siphon, to circulate the reaction mixture containing the catalyst in suspension and also to continuously remove the PO formed according to patent application WO 99/48883.

The presence of acetamide in the reaction medium at the end of the reaction has not been detected.

TABLE 2

| | | Example | | |
|---|---|---|---|---|
| | | 8 (ref) | 9 | 10 |
| Mol % CH₃CN/CH₃OH | | 0 | 0.3 | 1 |
| Degree of conversion of H₂O₂ | After 6 h | 64.6 | 60.8 | 55.0 |
| | After 30 h | 47.8 | 45.5 | 41.6 |
| Selectivity towards P0 | After 6 h | 85 | 90 | 92 |
| | After 30 h | 88 | 92 | 93 |

*= mol % PO/Σ (P0 + by-products)

1. Process for manufacturing an oxirane, in which an olefin is reacted, in a diluent chosen from water, alcohols and ketones, with a peroxide compound in the presence of a catalyst based on titanium silicalite of TS-1 type and in the presence of a nitrile.

2. Process according to claim 1, in which the molar ratio of the amounts of nitrile and of diluent used is at least 0.001% and not more than 50%.

3. Process according to claim 2, in which the molar ratio of the amounts of nitrile and of diluent used is at least 0.01% and not more than 10%.

4. Process according to any one of the preceding claims, in which the total amount of diluent and of nitrile used is at least 35% and not more than 99% by weight of the liquid phase containing the diluent, the nitrile, the dissolved olefin, the peroxide compound, a fraction of the oxirane formed and water.

5. Process according to any one of the preceding claims, in which the nitrile is chosen from linear or branched, saturated aliphatic nitrites and from aromatic nitriles, and the diluent is chosen from linear or branched, saturated aliphatic alcohols.

6. Process according to claim 5, in which the nitrile contains from 2 to 7 carbon atoms and the alcohol contains from 1 to 6 carbon atoms.

7. Process according to claim 6, in which the nitrile is acetonitrile and the diluent is methanol.

8. Process according to any one of the preceding claims, in which the olefin reacts with the peroxide compound in the presence of the catalyst, the diluent and the nitrile at a temperature of from 10° C. to 100° C. and at a pressure which may range from atmospheric pressure to 40 bar.

9. Process according to any one of the preceding claims, in which the oxirane is 1,2-epoxypropane, the olefin is propylene and the peroxide compound is hydrogen peroxide.

10. Process according to any one of claims 1 to 8, in which the oxirane is 1,2-epoxy-3-chloropropane, the olefin is allyl chloride and the peroxide compound is hydrogen peroxide.

11. A process for manufacturing an oxirane, in which an olefin is reacted, in a diluent chosen from water, alcohols and ketones, with a peroxide compound in the presence of a catalyst based on titanium silicalite of TS-1 type and in the presence of a nitrile.

12. The process according to claim 11, in which the molar ratio of the amounts of nitrile and of diluent used is at least 0.001% and not more than 50%.

13. The process according to claim 12, in which the molar ratio of the amounts of nitrile and of diluent used is at least 0.01% and not more than 10%.

14. The process according to claim 11, in which the total amount of diluent and of nitrile used is at least 35% and not more than 99% by weight of the liquid phase containing the diluent, the nitrile, the dissolved olefin, the peroxide compound, a fraction of the oxirane formed and water.

15. The process according to claim 11, in which the nitrile is chosen from linear or branched, saturated aliphatic nitrites and from aromatic nitrites, and the diluent is chosen from linear or branched, saturated aliphatic alcohols.

16. The process according to claim 15, in which the nitrile contains from 2 to 7 carbon atoms and the alcohol contains from 1 to 6 carbon atoms.

17. The process according to claim 16, in which the nitrile is acetonitrile and the diluent is methanol.

18. The process according to claim 11, in which the olefin reacts with the peroxide compound in the presence of the catalyst, the diluent and the nitrile at a temperature of from 10° C. to 100° C. and at a pressure which may range from atmospheric pressure to 40 bar.

19. The process according to claim 11, in which the oxirane is 1,2-epoxypropane, the olefin is propylene and the peroxide compound is hydrogen peroxide.

20. The process according to claim 11, in which the oxirane is 1,2-epoxy-3-chloropropane, the olefin is allyl chloride and the peroxide compound is hydrogen peroxide.
